(19) United States
(12) Patent Application Publication
(10) Pub. No.: US 2023/0066049 A1
(43) Pub. Date: Mar. 2, 2023

(54) PEPTIDES USEFUL IN PRESERVATION AND/OR RESTORATION OF FUNCTIONAL PANCREATIC ISLETS AND IN TREATING DIABETES

(71) Applicants: THE NATIONAL INSTITUTE FOR BIOTECHNOLOGY IN THE NEGEV LTD., Beer Sheva (IL); B. G. NEGEV TECHNOLOGIES AND APPLICATIONS LTD. AT BEN-GURION UNIVERSITY, Beer Sheva (IL)

(72) Inventor: Varda SHOSHAN-BARMATZ, Omer (IL)

(21) Appl. No.: 17/759,712

(22) PCT Filed: Jan. 26, 2021
(86) PCT No.: PCT/IL2021/050083

§ 371 Date: Jul. 28, 2022

Related U.S. Application Data
(60) Provisional application No. 62/966,582, filed on Jan. 28, 2020.

(51) Int. Cl. A61K 38/17; A61K 47/64; A61P 7/12

(52) U.S. Cl. CPC A61K 38/1709; A61K 47/644; A61P 7/12

(57) ABSTRACT

The present invention relates the field of preservation of functional pancreatic islet and treatment of diabetes, particularly to the use of peptides comprising analogues, particularly retro-analogues of VDAC1-derived peptides, for treating diabetes.

### FIELD OF THE INVENTION

The present invention relates to the field of preservation and/or number restoration of functional pancreatic islet and treatment of diabetes, particularly to the use of peptides comprising analogues, particularly retro-analogues of VDAC1-derived peptides, for treating diabetes.

### BACKGROUND OF THE INVENTION

Diabetes (Diabetes mellitus, DM) is a chronic metabolic disorder characterized by hyperglycemia (American Diabetes, A., Diagnosis and classification of diabetes mellitus. Diabetes Care, 2014. 37 Suppl 1: p. S81-90), with about 400 million individuals reported to suffer from Type 2 diabetes (T2D) worldwide, and approximately 320 million diagnosed with prediabetes (idf.org/action-on-diabetes/sdgs). DM is a result of insulin dysfunction, either due to defects in insulin secretion, defects in insulin action, or both (Kharroubi, A. T. and H. M. Darwish, Diabetes mellitus: The epidemic of the century. World J Diabetes, 2015. 6(6): p. 850-67). Insulin acts as the main anabolic hormone by binding the insulin receptor on target cells and propagating an intra-cellular response, mainly the fusion of vesicles containing the glucose transporter Glut4 to the plasma membrane, thereby enhancing cell glucose uptake (Tokarz, V. L., P. E. MacDonald, and A. Klip, J Cell Biol, 2018. 217(7): p. 2273-2289). Type 1 Diabetes (T1D) is characterized by insufficient insulin secretion due to complete destruction of the insulin producing β-cells of the pancreas (Daneman, D., 2006. 367(9513): p. 847-58; Devendra, D., E. Liu, and G. S. Eisenbarth, BMJ, 2004. 328(7442): p. 750-4), while T2D more commonly results from insulin resistance, namely a reduced response to insulin by the target tissues (Kharroubi, A. T. and H. M. Darwish, Diabetes mellitus: The epidemic of the century. World J Diabetes, 2015. 6(6): p. 850-67). The β-cells respond to insulin resistance by increased insulin production, although at some point the cells become exhausted with resulting hyperglycemia when the production of insulin does not match the increased insulin demand.

There is a high degree of association between T2D and non-alcoholic fatty-liver disease (NAFLD) and progressive, non-alcoholic steatohepatitis (NASH). A dysfunctional liver, such as seen in NAFLD, may dysregulate insulin action (Loomba, R., et al., Hepatology, 2012. 56(3): p. 943-51; Gastaldelli, A. and K. Cusi, JHEP Reports, 2019. 1(4): p. 312-328). Mitochondrial dysfunction has been implicated in both NAFLD and diabetes as well as in insulin resistance, which is shared by both conditions (Nassir, F. and J. A. Ibdah, Int J Mol Sci, 2014. 15(5): p. 8713-42; Garcia-Ruiz, C., et al., Free Radic Res, 2013. 47(11): p. 854-68; Sivitz, W. I. and M. A. Yorek, Antioxid Redox Signal, 2010. 12(4): p. 537-77). Dysfunctional mitochondria may further promote diabetes and NAFLD by impairing the energy homeostasis in hepatocytes or insulin target cells, thereby inducing an abnormal accumulation of lipids in hepatocytes or a reduced response to insulin (Chow, L., A. From, and E. Seaquist, Metabolism, 2010. 59(1): p. 70-85; Simoes, I. C. M., et al., Int J Biochem Cell Biol, 2018. 95: p. 93-99). It has been further suggested that the presence of NAFLD and progressive, non-alcoholic steatohepatitis (NASH) are associated with an increased incidence of T2D (Leite, N. C., et al., Liver Int, 2009. 29(1): p. 113-9). Often, NAFLD and T2D coexist and act synergistically to drive adverse clinical outcomes, where the presence of NAFLD increases the incidence of T2D and accelerates the development of diabetic complications (Adams, L. A., et al., Am J Gastroenterol, 2009. 104(4): p. 861-7; Shibata, M., et al., Diabetes Care, 2007. 30(11): p. 2940-4).

The voltage-dependent anion channel 1 (VDAC1), a multi-functional protein located in the outer mitochondrial membrane (OMM), is a key regulator of mitochondrial function. VDAC1 has been shown to serve as a mitochondrial gatekeeper, controlling the metabolic and energetic cross-talk between mitochondria and the rest of the cell, and is also one of the key proteins in mitochondria-mediated apoptosis. The importance of VDAC1 in cell energy and metabolism homeostasis is reflected by the findings that down-regulation of VDAC1 expression decreased metabolite exchange between mitochondria and the cytosol and inhibited cell growth (Shoshan-Barmatz, V. and D. Ben-Hail, VDAC, Mitochondrion, 2012. 12(1): p. 24-34; Shoshan-Barmatz, V., et al., Biochim Biophys Acta, 2015. 1848(10 Pt B): p. 2547-75; Shoshan-Barmatz, V., et al., Mol Aspects Med, 2010. 31(3): p. 227-85; Shoshan-Barmatz, V., et al., Biomolecules 10(11):1485). Under physiological conditions, VDAC1 is present as both a monomer and dimer, however, upon induction of apoptosis, the VDAC1 monomers/dimers undergo conformational changes to assemble into higher oligomeric states, forming a large channel that allows the release of pro-apoptotic proteins from the mitochondria. Thus, VDAC1 oligomerization has been demonstrated to play an important role in apoptosis by mediating cytochrome c (Cyto c) release, and to regulate apoptosis by binding apoptosis-regulating proteins (Keinan, N., D. Tyomkin, and V. Shoshan-Barmatz, Mol Cell Biol, 2010. 30(24): p. 5698-709; Zalk, R., et al., Biochem J, 2005. 386(Pt 1): p. 73-83; Abu-Hamad, S., et al., J Cell Sci, 2009. 122(Pt 11): p. 1906-16).

U.S. Pat. Nos. 8,119,601 and 8,648,045 to the inventor of the present invention and others disclose isolated VDAC1 derived peptides that are capable of inducing apoptosis in a cell and to pharmaceutical compositions comprising same useful in the treatment of diseases associated with aberrant apoptosis and cell hyperproliferation, particularly cancer. The peptides are derived from the N-terminal domain of VDAC1 as well as from VDAC1 β-strand 14 and its cytosolic β-loop.

U.S. Pat. No. 9,758,559 to the inventor of the present invention and others discloses short peptides based on the amino acids sequence of the N-terminal domain of

VDAC and to peptide conjugates further comprising a cell permeability enhancing moiety. The peptides, peptide conjugates and pharmaceutical composition comprising same are useful for treating diseases characterized by cell hyper-proliferation or resistance to cell death, particularly cancer.

The inventor of the present invention and co-workers has previously demonstrated that VDAC1 participates in diabetes-related dysfunction in the leptin deficient db/db mouse model of T2D (International Application Publication No. WO 2018/116307; Zhang, E., et al., Cell Metab, 2019. 29(1): p. 64-77 e6). In these mice, it was demonstrated that VDAC1 is overexpressed and mistargeted to the plasma membrane of insulin-secreting β-cells with loss of cellular ATP, and a consequent inhibition of depolarization-induced insulin secretion (Zhang et al., 2019, ibid). Treatment of db/db mice with a VDAC1 inhibitor, a piperazine molecule designated VBIT-4 (N-(4-chlorophenyl)-4-hydroxy-3-(4-(4-(trifluoromethoxy) phenyl) piperazin-l-yl) butanamide), International Application Publication No. WO 2017/046794; Ben-Hail, D., et al., J Biol Chem, 2016. 291(48): p. 24986-25003), prevented hyperglycemia, stimulated insulin secretion, and maintained normal glucose tolerance (Zhang et al., 2019, ibid).

International Application Publication No. WO 2017/037711 to the inventor of the present invention and others discloses peptides comprising analogues of VDAC-1 derived peptides having improved pharmacokinetics characteristics compared to native parent peptides, which are effective in impairing cell energy production, in inducing apoptosis and cell death, particularly of cancerous cells, in eliminating cancer stem cells and in reducing symptoms associated with fat accumulation in liver cells, particularly with nonalcoholic fatty liver disease (NAFLD) and symptoms associated thereto. In particular, a peptide comprising a retro-inverso analogue of a peptide derived from VDAC1 β-strand 14 and its cytosolic β-loop flanked by a tryptophan zipper, and a retro analogue of transferrin receptor binding domain (R-Tf-D-LP4) was found to reverse liver pathology to a normal-like state in a mouse model for NAFLD. Treatment with the R-Tf-D-LP4 peptide affected carbohydrate and lipid metabolism, and increased the expression of enzymes and factors associated with fatty acid transport to mitochondria, enhancing β-oxidation and thermogenic processes, while decreasing the expression of enzymes and regulators of fatty acid synthesis (WO 2017/037711; Pittala, S., et al., Mol Ther, 2019. (10): p. 1848-1862).

Publication of the inventor of the present invention and co-workers, published after the priority date of the present invention, describes that treatment with R-Tf-D-LP4 reduced blood glucose levels, and increased the number of insulin-producing cells in islets of diabetic (ob/ob) mice and in NAFLD affected mice. In addition, an increase in the number and size of the islets was observed, and the islet morphology was approved (Pittala S. et al., Cells, 2020. 9: p. 481; doi:10.3390/cells9020481)

There is still an unmet need for, and it would be highly advantageous to have novel methods for treating diabetes, particularly for preserving the function of β-cells and thereby maintaining normal glycemic levels and reducing the need for exogenous insulin administration.

### SUMMARY OF THE INVENTION

The present invention relates to the use of analogues of VDAC1-derived peptides for treating and/or preventing the progression of diabetes.

The present invention is based in part on the unexpected discovery that intravenous administration of a composition comprising a retro-inverso analogue of VDAC1-derived peptide (R-Tf-D-LP4) to obese (ob/ob) mice increased the number and improved the morphology of Langerhans islets of the mice pancreas, and reduced the blood glucose levels in these mice. ob/ob mice comprise a mutation in the gene responsible for the production of leptin and is an animal model of Type 2 diabetes. These finding have been further supported in mice in which administration of streptozotocin (STZ) and high-fat diet (STZ/HFD-32 mice model) lead to destruction of pancreatic β-cells leading to diabetes followed by NAFLD phenotypes. As in the obese mice, treatment with the retro-inverso analogue of VDAC1-derived peptide improved islet number and morphology and reduced blood glucose levels. In addition, increased insulin content within the islets and an increase in the expression of PDX1 transcription factor, necessary for pancreas differentiation and development, have been observed. This factor enhances the expression of insulin encoding gene and is essential for pancreas differentiation and development, β-cells maturation and maintenance and overall maintenance of glucose homeostasis in the pancreas. Without wishing to be bound by any specific theory or mechanism of action, the increase in the expression of this factor, observed mainly in β-cells of diabetic mice treated with the peptide analogous of the present invention, suggests that the reduction of blood glucose level in these mice is due to β-cell proliferation and increase in their number, leading to an increase in insulin production and an increase in insulin concentration. Treating diabetes via preservation of β-cell capability to produce and secrete insulin is most desirable in treating this widespread disease.

According to one aspect, the present invention provides a method for treating diabetes and/or preventing the progress of diabetes, comprising administering to a subject affected with prediabetes or with diabetes a therapeutically effective amount of a pharmaceutical composition comprising at least one synthetic peptide comprising a retro modified and partially or completely inverso modified analogue of a VDAC1-derived peptide, wherein the VDAC1-derived peptide comprises an amino acid sequence selected from the group consisting of SEQ ID NO:1 (KKLETAVNLAWTAGNSN) and SEQ ID NO:2 (MAVPPTYADLGKSARDVFTKGYGFGL).

According to certain embodiments the subject having diabetes is newly diagnosed early after onset of the disease. According to certain embodiments the diabetes is selected from the group consisting of Type 1 diabetes, Type 2 diabetes and gestational diabetes.

According to certain embodiments, the diabetes is Type 1 diabetes.

According to certain embodiments, the diabetes is Type 2 diabetes. According to certain embodiments, the diabetes is gestational diabetes.

According to certain embodiments, the subject affected with prediabetes or diabetes has a fasting plasma glucose (FPG) level of greater than 100 mg/dl, between about 100 and 130 mg/dl (Prediabetes), or greater than 130 mg/dl. According to certain additional or alternative embodiments, the subject affected with prediabetes or diabetes has a hemoglobin A1c (HbA1c) level above 6%.

According to certain embodiments, treating and/or preventing the progress of diabetes comprises reducing the FPG level and/or the HbA1c level of the subject by at least 5% compared to the FPG and/or HbA1c level measured before administration of the pharmaceutical composition of the invention.

According to certain embodiments, treating and/or preventing the progress of diabetes comprises at least one of preserving pancreatic islets number, size, and/or function; preventing pancreatic islet degeneration and/or dysfunction; restoring insulin secretion from pancreatic islet β-cells to a normal level; inducing glucose-stimulated insulin secretion; restoring the number of functional pancreatic islets to a normal level; and any combination thereof.

According to certain embodiments, the subject is a human subject at any age, including pre-pubertal child, post-pubertal child, adolescent and adult. Each possibility represents a separate embodiment of the present invention.

The pharmaceutical composition comprising the peptides of the invention typically further comprises a pharmaceutically acceptable excipient, diluent or carrier, and may be formulated for administration by a variety of routes including parenteral, oral, transdermal, topical, intranasal, or via a suppository.

According to certain embodiments, parenteral administration comprises intravenous, intramuscular, or intraperitoneal administration. According to certain exemplary embodiments, the pharmaceutical composition is administered intravenously.

The dosage and treatment frequency of the synthetic peptide to be administered will depends on the stage of the disease, its clinical manifestations, the subject gender, age, weight, general health and route of administration, and can be determined by standard clinical techniques as is known to one skilled in the art.

According to certain embodiments, the therapeutic amount of the peptide to be intravenously administered to a human subject is in the range of from about 1 to about 100 mg/Kg human body weight. According to certain embodiments, the peptide amount to be intravenously administered to a human subject is in the range of from about 1-75, 1-50, 1-25 mg/Kg human body weight. According to certain currently exemplary embodiments, the peptide intravenously administered to a human subject is at an amount of from about 1 to about 5 mg/Kg human body weight.

According to certain embodiments, the pharmaceutical composition comprising the peptides of the invention is administered at least once a day. According to some embodiments, the pharmaceutical composition is administered once a day. According to other embodiments, the pharmaceutical composition is administered once a week.

As used herein, the term “retro modified” refers to a peptide analogue which is composed of L-amino acids in which the amino acid residues are assembled in reversed direction in respect to the peptide from which it is retro modified.

As used herein, the term “inverso modified” refers to a peptide analogue which is composed of D-amino acids in which the amino acid residues are assembled in the same direction in respect to the peptide from which it is inverso modified. A partially inverso modified analogue refers to a peptide comprising at least one D-amino acid. A completely inverso modified analogue refers to a peptide made up of D-amino acids.

According to certain exemplary embodiments, the analogue is completely inverso modified.

According to certain embodiments, the VDAC1-derived peptide is designated LP4 and comprises the amino acid sequence set forth in SEQ ID NO:1 (KKLETAVNLAWTAGNSN). According to these embodiments, the sequence of the retro analogue comprises the amino acid sequence as set forth in SEQ ID NO:3 (NSNGATWALNVATELKK).

As used herein, the term “retro-inverso” analogue refers to a peptide analogue which is composed of D-amino acids in which the amino acid residues are assembled in the opposite direction with respect to the peptide from which it is retro-inverso modified.

According to certain exemplary embodiments, all the amino acids of the retro analogue as set forth in SEQ ID NO:3 are in D configuration forming a retro-inverso modified analogue of SEQ ID NO:1 having the sequence D-Asn-D-Ser-D-Asn-D-Gly-D-Ala-D-Thr-D-Trp-D-Ala-D-Leu-D-Asn-D-Val-D-Ala-D-Thr-D-Glu-D-Leu-D-Lys-D-Lys (SEQ ID NO:4).

According to certain embodiments, the VDAC1-derived peptide having the amino acid sequence set forth in SEQ ID NO:2, corresponding to amino acids 1-26 at the N-terminal of the native VDAC1. According to these embodiments, the sequence of the retro analogue comprises the amino acid sequence as set forth in SEQ ID NO:5 (LGFGYGKTFVDRAS KGLDAYTPPVAM).

According to certain exemplary embodiments, all the amino acids of the retro analogue as set forth in SEQ ID NO:5 are in D configuration forming a retro-inverso modified analogue of SEQ ID NO:2 having the sequence D-Leu-D-Gly-D-Phe-D-Gly-D-Tyr-D-Gly-D-Lys-D-Thr-D-Phe-D-Val-D-Asp-D-Arg-D-Ala-D-Ser-D-Lys-D-Gly-D-Leu-D-Asp-D-Ala-D-Tyr-D-Thr-D-Pro-D-Pro-D-Val-D-Ala-D-Met (SEQ ID NO:6).

According to certain embodiments, the synthetic peptide further comprises a cell penetrating and/or localization moiety. The localization moiety typically enhances the permeability of the synthetic peptide through the cell membranes. Any recognition and/or localization moiety as is known in the art can be used according to the teachings of the present invention, and it can be connected to any position of the analogue of VDAC1-derived peptide via a direct bond or via a spacer or linker. According to certain exemplary embodiments, the cell recognition and/or localization moiety is a peptide. According to some embodiments, the localization moiety is an intra-cellular localization peptide, also referred to as cell penetrating peptide (CPP).

According to some embodiments, the recognition and/or localization peptide is all L-stereomeric peptide. According to other embodiments, the recognition and/or localization peptide is all D-stereomeric peptide.

According to some embodiments, the recognition and/or localization peptide comprises transferrin-receptor binding domain (TO or a fragment thereof. According to certain exemplary embodiments, the transferrin-receptor binding domain comprises the amino acid sequence set forth in SEQ ID NO:7 (HAIYPRH). According to some embodiments, the Tf peptide consists of the amino acid sequence set forth in SEQ ID NO:7. According to certain exemplary embodiments, the recognition and/or localization peptide is a retro modified analogue of SEQ ID NO:7, having the amino acid sequence set forth in SEQ ID NO:8 (HRPYIAH). According to yet additional exemplary embodiments, the transferrin-receptor binding domain comprising the amino acids sequence set forth in any one of SEQ ID NO:7 and SEQ ID NO:8 is all L-stereomeric peptide.

According to other embodiments, the recognition and/or localization peptide is partially or completely inverso modified. According to some embodiments, the recognition and/or localization peptide is a completely inverso analogue of SEQ ID NO:7. According to certain exemplary embodiments, the recognition and/or localization peptide is a completely inverso modified analogue of SEQ ID NO:8.

According to additional certain embodiments, the recognition and/or localization peptide comprises the Drosophila antennapedia (Antp) domain or a fragment thereof. According to some embodiments, the Antp domain comprises the amino acid sequence set forth in SEQ ID NO:9 (RQIKIWFQNRRMKWKK) or a fragment thereof. According to other embodiments, the Antp domain consists of SEQ ID NO:9.

According to other embodiments, the recognition and/or localization peptide is a partially inverso modified analogue of SEQ ID NO:9 or of a part thereof. According to additional embodiments, the recognition and/or localization peptide is a completely inverso modified analogue of SEQ ID NO:9 or of a part thereof. According to further embodiments, the recognition and/or localization peptide is a retro modified analogue of SEQ ID NO:9, having the amino acid sequence set forth in SEQ ID NO:10 (KKWKMRRNQFWIKIQR) or a part thereof. According to yet additional embodiments, the recognition and/or localization peptide is an inverso analogue of SEQ ID NO:10.

According to some embodiments, the recognition and/or localization peptide is connected to the N-terminus of the analogue of VDAC1-derived peptide, directly or indirectly. Each possibility represents a separate embodiment of the present invention.

According to certain exemplary embodiments, the recognition and/or localization peptide is connected to the C-terminus of the analogue of VDAC1-derived peptide, directly or indirectly. Each possibility represents a separate embodiment of the present invention.

Most small peptides are flexible in solution and do not adopt the structure that the same sequence adopts in the native protein. Some of the VDAC1-derived peptides, according to VDAC1 topological model, exist in the form of β-loops. Therefore, according to certain embodiments the synthetic peptides of the invention comprises the amino acids sequences SWTWE (SEQ ID NO:11) and KWTWK (SEQ ID NO:12), together the “Tryptophan (Trp) zipper peptide”, each independently located at the C- or N-terminus of the analogue of the VDAC1 derived peptide. According to some embodiments, the Trp zipper peptide comprises a retro analogue of SEQ ID NO:11, said retro analogue has the amino acid sequence set forth in SEQ ID NO:13 (EWTWS). According to yet additional embodiments, the Trp zipper peptide comprises partially or completely an inverso peptide of any one of SEQ ID NOs:11-13. The Trp zipper peptide sequence can induce the formation of stable β-hairpins by tryptophan-tryptophan cross-strand pairs.

According to certain exemplary embodiments, the synthetic peptide comprises a peptide comprising the amino acid sequence set forth in any one of SEQ ID NO:1 and SEQ ID NO:2, wherein the peptide is retro modified and partially or completely inverso modified and wherein said peptide is flanked by Trp zipper amino acids at its N- and C-terminus.

According to certain exemplary embodiments, the synthetic peptide to be used with the methods of the invention comprises a retro-inverso analogue of SEQ ID NO:1 flanked by Trp zipper having the amino acids sequence set forth in SEQ ID NO:12 at its N-terminus and the amino acids sequence set forth in SEQ ID NO:13 at its C-terminus, further comprising a retro analogue of a recognition and/or localization peptide, having the amino acids sequence set forth in SEQ ID NO:7. According to certain currently exemplary embodiments, the synthetic peptide comprises the amino acids sequence set forth in SEQ ID NO:14 (D-Lys-D-Trp-D-Thr-D-Trp-D-Lys-D-Asn-D-Ser-D-Asn-D-Gly-D-Ala-D-Thr-D-Trp-D-Ala-D-Leu-D-Asn-D-Val-D-Ala-D-Thr-D-Glu-D-Leu-D-Lys-D-Lys-D-Glu-D-Trp-D-Thr-D-Trp-D-Ser-His-Arg-Pro-Tyr-Ile-Ala-His).

According to some embodiments, the peptide consists of the amino acids sequence set forth in SEQ ID NO:14.

It is to be understood that any combination of each of the aspects and the embodiments disclosed herein is explicitly encompassed within the disclosure of the present invention. Other objects, features and advantages of the present invention will become clear from the following description and drawings.

### BRIEF DESCRIPTION OF THE FIGURES

FIGS. 1A-1D demonstrate that treating ob/ob mice with R-Tf-D-LP4 peptide reduced the levels of blood glucose and decreased fat accumulation in 3T3-L1 adipocytes. FIG. 1A—ob/ob mice were untreated (●) or treated (▴) with intravenous injections of the R-Tf-D-LP4 peptide (14 mg/kg) three times a week and blood was drawn from an incision in the tail to measure glucose levels. The data represent the means±SD (n=6 mice). *p<0.05. The dashed line represents the blood glucose levels of wild type mice. FIG. 1B—Body weight of ob/ob mice untreated (●) or treated (▴) with the R-Tf-D-LP4 peptide (14 mg/kg) as followed for 7 weeks. FIG. 1C—The change in body weight as a function of time for mice untreated (●) or treated (▴) with the R-Tf-D-LP4 peptide (14 mg/kg) is presented. FIG. 1D—3T3-L1 cells were incubated with the 3, 10 or 20 μM of R-Tf-D-LP4 peptide for 24 h and then stained with Oil Red O and photographed, to visualize the lipid droplets stained in red. Quantification of Oil Red O stain was carried out by stain extraction with 100% isopropanol for 5 min and measurement of the absorbance at 492 nm. The results are means±SEM (n=3; ***p≤0.001).

FIGS. 2A-2B demonstrate that ob/ob mice show large islets, and an increase in the islet number by treatment with the R-Tf-D-LP4 peptide. ob/ob mice were untreated or treated with intravenous injections of the R-Tf-D-LP4 peptide (14 mg/kg) three times a week, mice were scarified, and the pancreases were removed and fixed. Representative paraffin-embedded, formaldehyde-fixed, pancreatic sections were stained with hematoxylin eosin (FIG. 2A) or immunofluorescence (IF) stained with anti-insulin antibodies and with DAPI staining of the nuclei (FIG. 2B). Islets are indicated by arrows (FIG. 2A) or circled by dashed line (FIG. 2B).

FIGS. 3A-3B demonstrate the effect of R-Tf-D-LP4 on the Langerhans islets in STZ/HFD-32 fed mice. FIG. 3A—Representative formaldehyde-fixed, paraffin-embedded pancreas sections from chow-diet fed mice of from STZ/HFD-32-fed mice at the steatosis stages without (control) and with R-Tf-D-LP4 peptide treatment. The sections were IF stained with anti-insulin antibodies and the nucleus were stained with DAPI. Arrows point to disrupted islets and circled arrows point to small non-disrupted islets. FIG. 3B—Pancreatic sections from chow-diet fed, STZ/HFD-32-fed mice without and with R-Tf-D-LP4 peptide treatment at the NASH stage were stained with H & E. The islets are circled.

FIGS. 4A-4C show glucagon and glucose transporter-2 (Glut-2) staining of Langerhans islets in STZ/HFD-32 fed mice at the steatosis or NASH stage and the effect of R-Tf-D-LP4 peptide treatment. Representative formaldehyde-fixed, paraffin-embedded, pancreases sections from two mice from each of the experimental groups (chow-diet fed, and STZ/HFD-32-fed mice without and with R-Tf-D-LP4 peptide treatment) at the steatosis (FIG. 4A) or NASH stage (FIG. 4B) were IHC stained with anti-glucagon antibodies. Arrows point to the islets. FIG. 4C—Representative formaldehyde-fixed, paraffin-embedded, pancreatic sections from chow-diet fed mice and STZ/HFD-32-fed mice at steatosis without and with R-Tf-D-LP4 peptide treatment, IF stained with anti-Glut-2 antibodies with nuclei stained with DAPI.

FIGS. 5A-5C show VDAC1 expression level in Langerhans islets in mice fed with regular (chow) diet and STZ/HFD-32-fed mice untreated (control) or treated with R-Tf-D-LP4. FIG. 5A—Pancreatic sections from chow-diet fed mice and from STZ/HFD-32-fed mice untreated (control) or treated with R-Tf-D-LP4 peptide (14 mg/kg) were IHC stained for VDAC1 using specific antibodies followed by hematoxylin staining. FIG. 5B—A quantitative analysis of VDAC1 staining intensity using a panoramic microscope and HistoQuant software (Quant Center 2.0 software, 3DHISTECH Ltd). Results are means±SEM (n=3-5; ***p≤0.001). FIG. 5C Representative paraffin-embedded, formaldehyde-fixed pancreatic sections from chow-diet fed mice and from STZ/HFD-32-fed mice without and with R-Tf-D-LP4 peptide treatment at the steatosis stage were IF co-stained with anti-VDAC1 and anti-insulin antibodies and the nuclei were stained with DAPI.

FIGS. 6A-6B demonstrate the effect of R-Tf-D-LP4 on the expression levels of the proliferation marker Ki-67 in pancreas of STZ/HFD-32 fed mice. FIG. 6A—Sections from two mice from each of the experimental groups (chow-diet fed mice, STZ/HFD-32-fed mice without (control) and with R-Tf-D-LP4 peptide treatment) at the steatosis stage, were IF stained with anti-Ki-67 antibodies and with DAPI for nuclear staining. The islets are circled and the arrows point to Ki-67 staining of cells located around the periphery or inside the Langerhans islets. FIG. 6B—Ki-67 positive cell inside and around the edge of the Langerhans islets were counted in the whole sections. Results are means±SEM (n=3-5 pancreases; *p≤0.05).

FIGS. 7A-7B show that PDX1 expression is increased in islets of Langerhans by R-Tf-D-LP4 peptide treatment. FIG. 7A—Representative pancreatic sections from 3-5 mice from each experimental group (chow-diet fed mice, STZ/HFD-32-fed mice, and STZ/HFD-32 fed mice treated with R-Tf-D-LP4 peptide) were IF stained with anti-PDX1 or anti-insulin antibodies as indicted and nuclei stained with DAPI. FIG. 7B—Similar experiment with sections IF stained with anti-PDX1 or anti-glucagon antibodies as indicated and nuclei stained with DAPI. Enlargement of selected image area are presented to clearly demonstrate that PDX1 was also localized in the nucleus.

### DETAILED DESCRIPTION OF THE INVENTION

The present invention answers a long felt need for an effective treatment of diabetes via preservation and/or restoration of Langerhans islets and the insulin-producing beta-cells therein. The present invention shows that unexpectedly, a synthetic analogue of a peptide derived from VDAC1 was highly effective in improving the state of the Langerhans islets with respect to size, number, and insulin production in obese model mice (ob/ob, Lindström P. Scientific World Journal. 7: 666-85, 2007), and accordingly in restoring the blood glucose levels to close to normal. These findings have been further validated in mice induced to have steatosis NASH and Type 2 diabetes phenotypes (designated herein STZ/HFD-32 mice or STZ/HFD-32-fed mice, Fujii, M., et al., Med Mol Morphol, 2013. 46(3): p. 141-52). Preserving and/or restoring the intrinsic ability of Langerhans islet to produce insulin is of high significance as it may prevent the progression of prediabetes to diabetes and improve the disease prognosis, and further as it enables to reduce the frequency and/or amounts of exogenous insulin administration, being a burden on diabetic patients.

### Definitions

The term “prediabetes” as used herein refers to a borderline diabetes, which is usually a precursor to diabetes. It occurs when the blood glucose levels are higher than normal, but not high enough for the patient to be considered to have diabetes. It is often described as the “grey area” between normal blood sugar and diabetic levels. Pre-diabetes may be also referred to as impaired fasting glucose (IFT), if a patient has higher than normal sugar levels after a period of fasting, or as impaired glucose tolerance (IGT), if a patient has higher than normal sugar levels following eating.

The terms “diabetes” or “diabetic disorder” or “diabetes mellitus” as used interchangeably herein, refer to a disease which is marked by elevated levels of sugar (glucose) in the blood (hyperglycemia). Diabetes can be caused by insufficient amount of insulin, resistance to insulin, or both. Diabetes includes the two most common types of the disorder, namely Type 1 diabetes and Type 2 diabetes, which both result from the body's inability to regulate insulin. Insulin is a hormone released by the pancreas in response to increased levels of glucose in the blood. Overtime, diabetes leads to serious damage to the heart, blood vessels, eyes, kidneys and nerves.

The terms “Type 1 diabetes”, “Type 1 diabetes mellitus” and “T1D” are used herein interchangeably, referring to a chronic disease that occurs when the pancreas produces too little insulin to regulate blood sugar levels appropriately. Type 1 diabetes is also referred to as insulin-dependent diabetes mellitus, IDDM, and diabetes-type I. The disease often affects young children and thus also referred to as juvenile onset diabetes. Similar T1D that affects older individuals is called “late onset” type 1 diabetes. T1D is the result of a progressive autoimmune destruction of the pancreatic β-cells with subsequent insulin deficiency.

The terms “Type 2 diabetes”, “Type 2 diabetes mellitus” and “T2D” are used herein interchangeably, referring to a chronic disease, typically affecting adults, that ranges from predominant insulin resistance with relative insulin deficiency to prevailing defective secretion with insulin resistance, resulting in chronic abnormally high level of glucose present in the blood of a subject.

Normal fasting plasma glucose (FPG) levels are considered as less than about 100 mg/dl, between about 100 and 126-130 mg/dl for impaired glucose metabolism, and greater than about 126-130 mg/dl for diabetics.

The terms “gestational diabetes” and “gestational diabetes mellitus (GDM)” as used herein refer to any degree of glucose intolerance with onset or first recognition during pregnancy. The definition applies whether insulin or only diet modification is used for treatment and whether or not the condition persists after pregnancy. Gestational diabetes affects 3-9% of pregnancies, depending on the population studied. Gestational diabetes can occur during pregnancy because of insulin resistance or reduced production of insulin.

The terms “VDAC1” and “hVDAC1” are used herein interchangeably and refer to the human voltage-depended anion channel isoform 1 (hVDAC1) of a highly conserved family of mitochondrial porins. Four VDAC isoforms, encoded by three genes, are known to date; as used herein, the terms “VDAC1” and human “hVDAC1” refer to a 283 amino acid protein (NP_003365).

The term “peptide” as used herein is meant to encompass natural, non-natural and/or chemically modified amino acid residues, each residue being characterized by having an amino and a carboxy terminus, wherein two or more amino acids are connected one to the other by peptide or non-peptide bonds. The amino acid residues are represented throughout the specification and claims by either one or three-letter codes, as is commonly known in the art. In the sequence listing of the invention, conventional amino acid residues have their conventional meaning. Specific peptides of the present invention are preferably utilized in β-hairpin form.

As used herein the term “about” refers to any numerical value ±10%.

According to one aspect, the present invention provides a method for treating and/or preventing the progress of diabetes, comprising administering to a subject affected with prediabetes or with diabetes a therapeutically effective amount of a pharmaceutical composition comprising at least one synthetic peptide comprising a retro modified and partially or completely inverso modified analogue of a VDAC1-derived peptide, wherein the VDAC1-derived peptide comprises an amino acid sequence selected from the group consisting of SEQ ID NO:1 (KKLETAVNLAWTAGNSN) and SEQ ID NO:2 (MAVPPTYADLGKSARDVFTKGYGFGL).

According to additional aspect, the present invention provides a pharmaceutical composition comprising at least one synthetic peptide comprising a retro modified and partially or completely inverso modified analogue of a VDAC1-derived peptide, wherein the VDAC1-derived peptide comprises an amino acid sequence selected from the group consisting of SEQ ID NO:1 (KKLETAVNLAWTAGNSN) and SEQ ID NO:2 (MAVPPTYADLGKSARDVFTKGYGFGL), for use in treating and/or preventing the progress of diabetes in a subject affected with prediabetes or with diabetes.

According to certain embodiments, the subject having diabetes is newly diagnosed for diabetes. According to certain exemplary embodiments, the subject is newly diagnosed early after onset of the disease. According to certain embodiments, the subject has severe hyperglycemia. According to certain embodiments the diabetes is selected from the group consisting of T1D, T2D and gestational diabetes.

According to certain embodiments, the diabetes is Type 1 diabetes.

According to certain embodiments, the diabetes is Type 2 diabetes.

According to certain embodiments, the diabetes is gestational diabetes.

According to certain embodiments, the subject affected with prediabetes or diabetes has a fasting plasma glucose (FPG) level of greater than 100 mg/dl, between about 100 and 130 mg/dl, or greater than 130 mg/dl. According to certain additional or alternative embodiments, the subject affected with prediabetes or diabetes has a hemoglobin A1c (HbA1c) level above 6%.

The synthetic peptides to be used are as described hereinabove.

The term “treating” as used herein refers to inhibiting the disease state, i.e., arresting the development of the disease state or its clinical symptoms, or relieving the disease state, i.e., causing temporary or permanent regression of the disease state or its clinical symptoms. The term is interchangeable with any one or more of the following: abrogating, ameliorating, inhibiting, attenuating, blocking, suppressing, reducing, halting, alleviating or preventing symptoms associated with the disease.

According to certain embodiments, the term “treating” refers to lowering or reducing glucose in a subject having FPG levels greater than 100 mg/dl, for example, between about 100 and 130 mg/dl, or greater than 130 mg/dl, or from between about 100 and 126 mg/dl, or grater that 126 mg/dl, by at least about 5%, for example by about 5-10%, about 10-20%, about 20-30%, or about 30-50%, or more, or for example from greater than 200 mg/dl to less than 200 mg/dl, from greater than 150 mg/dl to less than 150 mg/dl, from greater than 130, 129, 128, 127, 126 or 125 mg/dl to less than 130, 129, 128, 127, 126 or 125 mg/dl, etc., by administering to the subject a pharmaceutical composition comprising at least one peptide of the invention. Alternatively, or in addition, “treating” refers to lowering or reducing glucose in subjects with baseline HbA1c levels greater than about 5%, 6%, 7%, 8%, 9% or 10%, in particular 5%, 6%, or 7%.

According to certain embodiments, preventing the progress of diabetes comprises preventing the progression of pre-diabetes to diabetes.

According to certain embodiments, treating and/or preventing the progress of diabetes comprises at least one of preserving pancreatic islets number, size, and/or function at a normal level or about a normal level; preventing pancreatic islet degeneration and/or dysfunction; restoring insulin secretion from pancreatic islet β-cells to a normal level or about a normal level; inducing glucose-stimulated insulin secretion; restoring the number of functional pancreatic islets to a normal level or about a normal level; and any combination thereof.

The terms “retro-modified” and “inverso-modified” are used herein according to their meaning in the art and as defined hereinabove.

As used herein, the term “retro-inverso modified” refers to a peptide analogue which is composed of D-amino acids, wherein the amino acid residues are assembled in the opposite direction in respect to the peptide from which it is retro-inverso modified.

According to certain embodiments, the VDAC1-derived peptide comprises the amino acid sequence set forth in any one of SEQ ID NO:1 and SEQ ID NO:2. According to certain embodiments, the VDAC1-derived peptide comprises the amino acid sequence set forth in SEQ ID NO:1. According to certain additional embodiments, the VDAC1-derived peptide comprises the amino acid sequence set forth in SEQ ID NO:2.

According to certain embodiments, the VDAC1-derived peptide consists of the amino acid sequence set forth in any one of SEQ ID NO:1 and SEQ ID NO:2. According to certain embodiments, the VDAC1-derived peptide consists of the amino acid sequence set forth in SEQ ID NO:1. According to certain additional embodiments, the VDAC1-derived peptide consists of the amino acid sequence set forth in SEQ ID NO:2.

According to certain exemplary embodiments, the analogue of VDAC1-derived peptide is a retro-inverso analogue with respect to SEQ ID NO: 1. According to these embodiments, the analogue of VDAC1 derived peptide comprises the amino acids sequence set forth in SEQ ID NO:3, wherein all the amino acids are replaced with D-amino acids to comprise SEQ ID NO:4.

According to certain embodiments, the analogue of VDAC1 derived peptide consists of the amino acids sequence set forth in SEQ ID NO:3, wherein all the amino acids are D-amino acids to form SEQ ID NO:4.

According to certain additional exemplary embodiments, the analogue of VDAC1-derived peptide is retro-inverso analogue with respect to SEQ ID NO:2. According to these embodiments, the analogue of VDAC1-derived peptide comprises the amino acid sequence set forth in SEQ ID NO:5, wherein all the amino acids are replaced with D-amino acids to comprise SEQ ID NO:6.

According to certain embodiments, the analogue of VDAC1-derived peptide consists of the amino acid sequence set forth in SEQ ID NO:5, wherein all the amino acids are D-amino acids to form SEQ ID NO:6.

According to certain embodiments, the synthetic peptide further comprises a cell recognition and/or localization moiety. According to certain exemplary embodiments, the recognition and/or localization moiety is a peptide moiety enhancing penetration into cells. Such peptides, typically referred to as Cell Penetrating Peptides (CPPs), consists of short peptide sequences that rapidly translocate molecules, including large molecules, into the cell interior in a seemingly energy- and sometimes receptor-independent manner. CPPs have low toxicity and a high yield of delivery. Exemplary CPPs are the transferrin-receptor binding domain (Tf) (having the amino acid sequence set forth in SEQ ID NO:7), Antp domain (having the amino acid sequence set forth in SEQ ID NO:9), the HIV-1 transcriptional factor TAT, and VP22 from HSV-1.

According to certain exemplary embodiments, the recognition and/or localization peptide comprises SEQ ID NO:7 or a retro analogue thereof, having the amino acid sequence set forth in SEQ ID NO:8. According to additional exemplary embodiments, the recognition and/or localization peptide consist of SEQ ID NO:7 or a retro analogue thereof, consisting of the amino acid sequence set forth in SEQ ID NO:8. According to other embodiments, the recognition and/or localization peptide is a partially inverso modified analogue of SEQ ID NO:7 or of SEQ ID NO:8. According to additional embodiments, the recognition and/or localization peptide is a completely inverso modified analogue of SEQ ID NO:7 or of SEQ ID NO:8.

According to certain additional exemplary embodiments, the recognition and/or localization peptide comprises SEQ ID NO:9 or a retro analogue thereof, having the amino acid sequence set forth in SEQ ID NO:10. According to additional exemplary embodiments, the recognition and/or localization peptide consist of SEQ ID NO:9 or a retro analogue thereof, consisting of the amino acid sequence set forth in SEQ ID NO:10. According to other embodiments, the recognition and/or localization peptide is a partially inverso modified analogue of SEQ ID NO:9 or of SEQ ID NO:10. According to additional embodiments, the recognition and/or localization peptide is a completely inverso modified analogue of SEQ ID NO:9 or of SEQ ID NO:10.

According to certain embodiments, the synthetic peptides of the invention further comprise the amino acids sequences SWTWE (SEQ ID NO:11) and KWTWK (SEQ ID NO:12), together the “Tryptophan (Trp) zipper peptide” or retro analogue(s) thereof, each independently located at the C- or N-terminus of the analogue of VDAC1 derived peptide.

According to certain embodiments, the Trp zipper peptide or a retro-analogue thereof is all L-stereomeric peptide. According to other embodiments, the Trp zipper peptide or a retro-analogue thereof is partially inverso modified. According to additional embodiments, the Trp zipper peptide or a retro-analogue thereof is completely inverso modified containing only D-amino acids. According to some embodiments, the retro-analogue of the Trp zipper peptide comprises the amino acid sequence set forth in SEQ ID NO:13 (EWTWS). According to yet additional embodiments, the Trp zipper peptide comprises retro-inverso analogue of SEQ ID NO:11, SEQ ID NO:12 or the combination thereof.

The term “all L-stereomeric peptide” as used herein refers to a peptide in which all the amino acids are L-amino acids. The term “all D-stereomeric peptide” as used herein refers to a peptide in which all the amino acids are D-amino acids.

According to additional exemplary embodiments, the synthetic peptide to be used with the methods and teachings of the invention comprises a retro-inverso analogue of SEQ ID NO:1 flanked by Trp zipper having the amino acids sequence set forth in SEQ ID NO:12 at its N-terminus and the amino acids sequence set forth in SEQ ID NO:13 at its C-terminus, further comprising a recognition and/or localization peptide having the amino acids sequence set forth in SEQ ID NO:8. According to some embodiments, the synthetic peptide comprises the amino acids sequence set forth in SEQ ID NO:16 (Lys-Trp-Thr-Trp-Lys-D-Asn-D-Ser-D-Asn-D-Gly-D-Ala-D-Thr-D-Trp-D-Ala-D-Leu-D-Asn-D-Val-D-Ala-D-Thr-D-Glu-D-Leu-D-Lys-D-Lys-Glu-Trp-Thr-Trp-Ser-His-Arg-Pro-Tyr-Ile-Ala-His). According to certain exemplary embodiments, the peptide consists of SEQ ID NO:16.

An exemplary sequence of a synthetic peptide to be used with the methods and teachings of the invention, referred to herein interchangeably as Retro-Tf-D-LP4, R-Tf-D-LP4, retro-inverso peptide, retro-inverso Tf-D-LP4 or retro-inverse Tf-D-LP4, comprises from the N-to C-terminus a Trp zipper peptide having the amino acid sequence set forth in SEQ ID NO:12, wherein the amino acids are D-amino acids, followed by a retro-inverso analogue of SEQ ID NO:1 (having the amino acid sequence of SEQ ID NO:3 wherein the amino acids are D-amino acids to form SEQ ID NO:4) followed by a Trp zipper peptide having the amino acid sequence set forth in SEQ ID NO:13, wherein the amino acids are D-amino acids, followed by a retro-analogue of a the Tf recognition and/or localization peptide having the amino acids sequence set forth in SEQ ID NO:7 (the retro analogue having the amino acid sequence set forth in SEQ ID NO:8). According to certain embodiments, the peptide, consists of the amino acid sequence D-Lys-D-Trp-D-Thr-D-Trp-D-Lys-D-Asn-D-Ser-D-Asn-D-Gly-D-Ala-D-Thr-D-Trp-D-Ala-D-Leu-D-Asn-D-Val-D-Ala-D-Thr-D-Glu-D-Leu-D-Lys-D-Lys-D-Glu-D-Trp-D-Thr-D-Trp-D-Ser-His-Arg-Pro-Tyr-Ile-Ala-His (SEQ ID NO:14).

An additional exemplary sequence of a synthetic peptide to be used with the methods and teachings of the invention comprises from the N-to C-terminus a retro analogue of Antp cell penetration peptide having the amino acids sequence set forth in SEQ ID NO:9 (the retro analogue having the amino acid sequence set forth in SEQ ID NO:10) followed by a retro-inverso analogue of SEQ ID NO:2 (having the amino acid sequence of SEQ ID NO:5 wherein the amino acids are D-amino acids to form SEQ ID NO:6). The peptide, referred to herein as “retro-inverso N-terminal” or Retro-D-N-Ter comprises the amino acid sequence Lys-Lys-Trp-Lys-Met-Arg-Arg-Asn-Gln-Phe-Trp-Ile-Lys-Ile-Gln-Arg-D-Leu-D-Gly-D-Phe-D-Gly-D-Tyr-D-Gly-D-Lys-D-Thr-D-Phe-D-Val-D-Asp-D-Arg-D-Ala-D-Ser-D-Lys-D-Gly-D-Leu-D-Asp-D-Ala-D-Tyr-D-Thr-D-Pro-D-Pro-D-Val-D-Ala-D-Met (SEQ ID NO:15).

According to certain embodiments, the C-terminus of the peptides of the invention may be amidated, acylated, reduced or esterified. Each possibility represents a separate embodiment of the present invention.

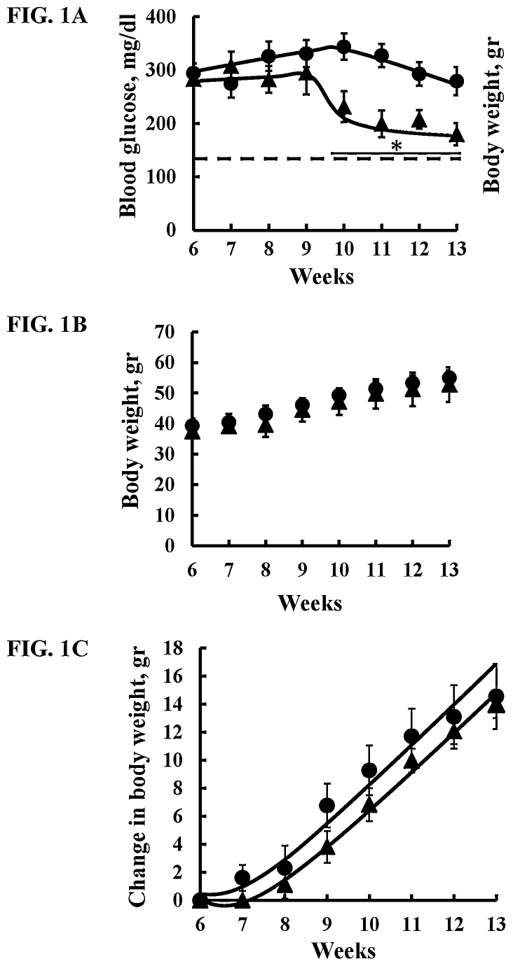

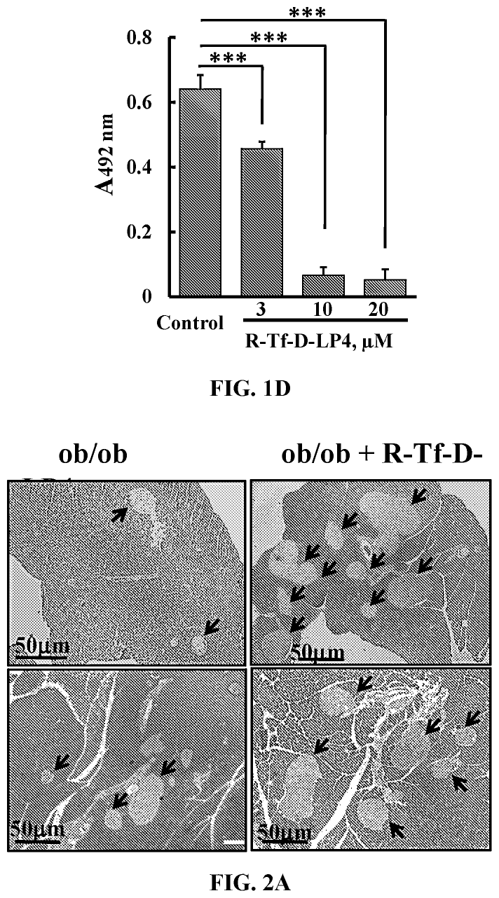

Unexpectedly, as exemplified hereinbelow, the Retro-Tf-D-LP4 is highly active in restoring the blood glucose levels to close to normal and improved the state of the Langerhans islets with respect to size, number, and insulin production. The results presented below of insulin stained islets, suggest that R-Tf-D-LP4 peptide treatment of the ob/ob and STZ/HFD-32-fed mice induces 13-cell production (FIGS. 1, 2, 3, Table 2).

Thus, according to a further aspect, the present invention provides a method for at least one of preserving pancreatic islets number and/or size and/or function; preventing pancreatic islet degeneration and/or dysfunction; restoring insulin secretion from pancreatic islet β-cells; inducing glucose-stimulated insulin secretion; restoring the number of functional pancreatic islets to a normal level; and any combination thereof, comprising administering to a subject affected with prediabetes or with diabetes a therapeutically effective amount of a pharmaceutical composition comprising at least one synthetic peptide comprising a retro modified and partially or completely inverso modified analogue of a VDAC1-derived peptide, wherein the VDAC1-derived peptide comprises an amino acid sequence selected from the group consisting of SEQ ID NO:1 and SEQ ID NO:2.

According to yet additional aspect, the present invention provides a pharmaceutical composition comprising at least one synthetic peptide comprising a retro modified and partially or completely inverso modified analogue of a VDAC1-derived peptide, wherein the VDAC1-derived peptide comprises an amino acid sequence selected from the group consisting of SEQ ID NO:1 and SEQ ID NO:2, for use is at least one of preserving pancreatic islets number and/or size and/or function; preventing pancreatic islet degeneration and/or dysfunction; restoring insulin secretion from pancreatic islet β-cells; inducing glucose-stimulated insulin secretion; restoring the number of functional pancreatic islets to a normal level; and any combination thereof, in a subject affected with prediabetes or diabetes.

The islets of Langerhans are comprised of glucagon-producing α-cells located in the periphery of the islet, insulin-producing β-cells in the interior, and somatostatin-producing γ-cells that are evenly distributed across the islet (Wilcox, G., Clin Biochem Rev, 2005. 26(2): p. 19-39; Steiner, D.J., et al., Islets, 2010. 2(3): p. 135-45).

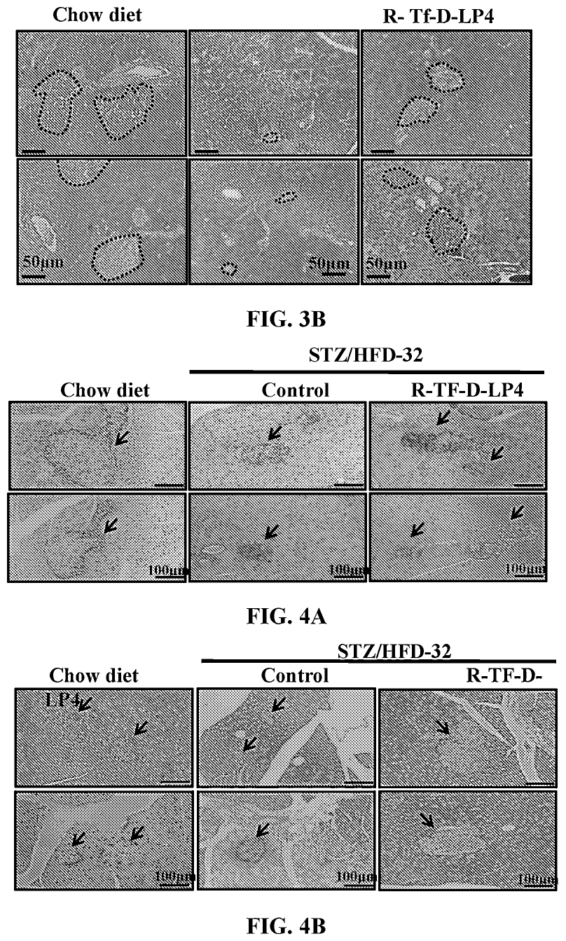

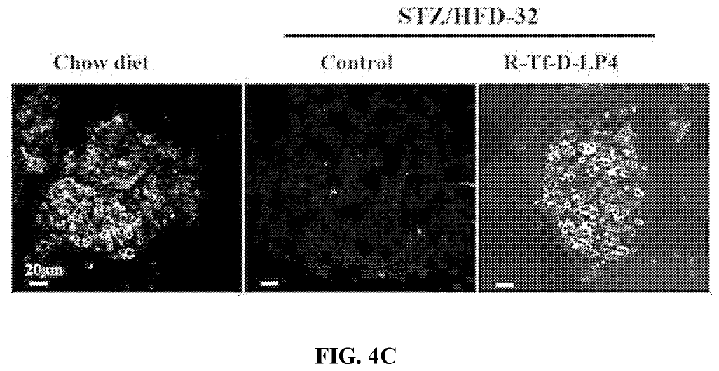

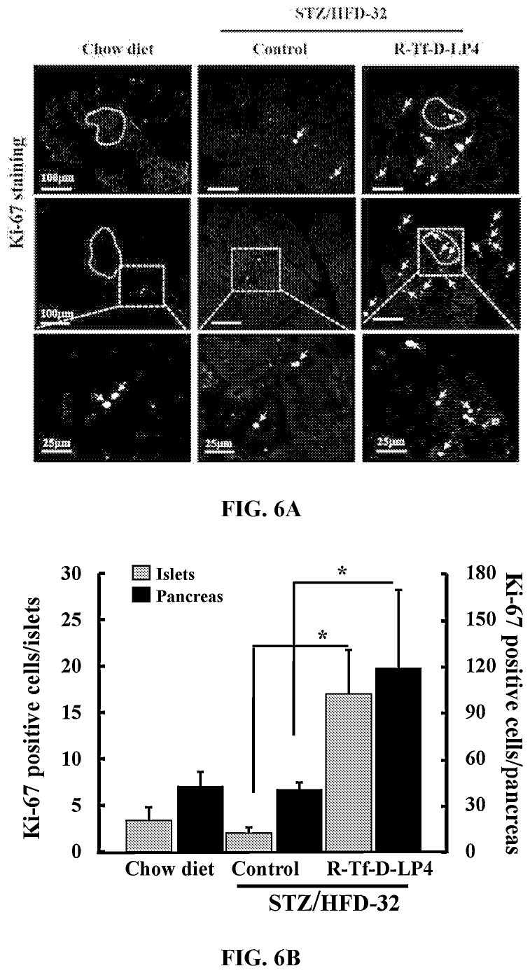

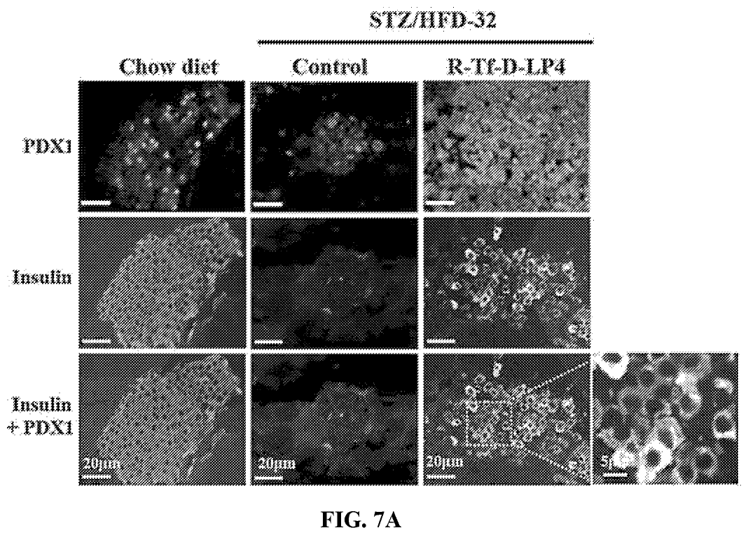

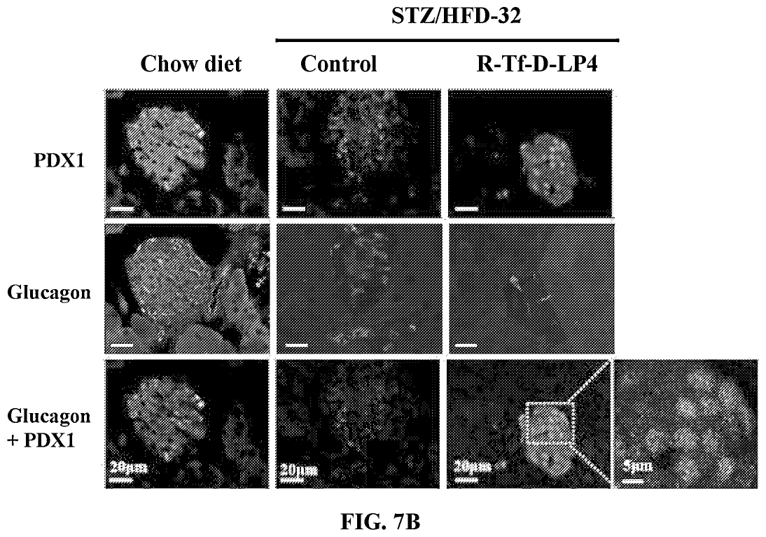

To investigate the source of the observed increased number and size of the β-cell containing islets after treatment of STZ/HFD-32-fed mice with the R-Tf-D-LP4 peptide, the expression of insulin and glucagon was examined (FIGS. 3, 4, Table 2), as well as the proliferation factor Ki-67 (FIG. 6) and the transcription factor PDX1 (pancreatic and duodenal homeobox 1, also known as insulin promoter factor 1) (FIG. 7). The results demonstrated high numbers of Ki-67 positive cells both inside and outside of the islets of STZ/HFD-32-fed mice treated with the R-Tf-D-LP4-peptide (FIG. 6). This implies that the observed increase in β-cell content in the islets of these mice is due to increased proliferation of cells within the islets, but also of other cells outside of the islets that can explain the increase in the number of small islets (FIG. 3, Table 2).

PDX1 has a well described role in the function and survival of β cells where it is a key regulator of normal pancreatic development and β cell differentiation, inducing differentiation of both embryonic stem cells and bone-marrow-derived mesenchymal stem cells into insulin-producing cells (Jurczyk, A., R. Bortell, and L. C. Alonso, Curr Opin Endocrinol Diabetes Obes, 2014. 21(2): p. 102-8; Habener, J. F. and V. Stanojevic, alpha-cell role in beta-cell generation and regeneration. Islets, 2012. 4(3): p. 188-98; Brissova, M., et al., J Biol Chem, 2002. 277(13): p. 11225-32). PDX1 is transiently expressed during the development of the pancreas and duodenum and in the differentiation and maturation of β-cells, and acts as an enhancer for several genes including the insulin-transcribing gene (Ahlgren, U., et al., Genes Dev, 1998. 12(12): p. 1763-8). Thus, in the adult pancreas, PDX1 is responsible for the regulation of genes that are essential for islet development, function, proliferation, and maintenance of glucose homeostasis (Babu, D. A., T. G. Deering, and R. G. Mirmira, A feat of metabolic proportions: Mol Genet Metab, 2007. 92(1-2): p. 43-55; Khoo, C., et al., Research resource: the pdx1 cistrome of pancreatic islets. Mol Endocrinol, 2012. 26(3): p. 521-33). Accordingly, downregulating PDX1 expression in humans and in animal models results in type 2 diabetes, β cell dysfunction, and impaired islet compensation in the presence of insulin resistance (Park, J. H., et al., J Clin Invest, 2008. 118(6): p. 2316-24).

The role of PDX1 in β cell survival in response to HFD has already been established (Sachdeva, M. M., et al., Proc Natl Acad Sci U S A, 2009. 106(45): p. 19090-5). While PDX1 expression is not itself induced by stress, it regulates islet compensation for HFD-induced insulin resistance, in part through direct transcriptional regulation of Atf4 and Wsf1 (Sachdeva et al., 2009, ibid). Here, co-staining the pancreas of STZ/HFD-32 fed mice with anti-PDX1 and anti-insulin antibodies, or with anti-PDX1 and anti-glucagon antibodies revealed a significant increase of PDX1 expression in mice fed with STZ/HFD-32 diet treated with the R-Tf-D-LP4 peptide (FIG. 7). PDX1 expression was mainly in the β-cells, suggesting that the source of the increased number of β-cells upon treatment with the R-Tf-D-LP4 peptide is most likely due to proliferation of β-cells. However, the number of Ki-67 expressing cells was increased upon treatment of STZ/HPD-32-fed mice with the R-Tf-D-LP4 peptide not only in the islets but also in cells outside of the islets (FIG. 6). This may suggest that other cell types undergo differentiation into β-cells, resulting in an increase in the number of new small islets (FIG. 3 and Table 2). Identification of these 62 -cells precursor will require additional study.

The peptides of the present invention are administered to the subject affected with diabetes or prediabetes within a pharmaceutical composition, typically further comprising pharmaceutically acceptable excipients, diluents or carriers.

As used herein, “pharmaceutical” will be understood to encompass both human and animal pharmaceuticals. Useful carriers include, for example, water, acetone, ethanol, ethylene glycol, propylene glycol, butane-1, 3-diol, isopropyl myristate, isopropyl palmitate, or mineral oil. Methodology and components for formulation of pharmaceutical compositions are well known, and can be found, for example, in Remington's Pharmaceutical Sciences, Eighteenth Edition, A. R. Gennaro, Ed., Mack Publishing Co. Easton Pa., 1990.

Apart from other considerations, the fact that the novel active ingredients of the invention are peptides, peptide analogs or peptidomimetics, dictates that the formulation be suitable for delivery of these types of compounds. Pharmaceutical compositions typically comprise a therapeutically effective amount of at least one of the peptide sequences of the invention, including subsequences, variants and modified forms of the exemplified peptide sequences and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that could be used in the pharmaceutical compositions and dosage forms used in the invention. Typical buffers include, but are not limited to pharmaceutically acceptable weak acids, weak bases, or mixtures thereof. Buffer components also include water soluble materials such as phosphoric acid, tartaric acids, lactic acid, succinic acid, citric acid, acetic acid, ascorbic acid, aspartic acid, glutamic acid, and salts thereof.

A primary solvent in a vehicle may be either aqueous or non-aqueous in nature. In addition, the vehicle may contain other pharmaceutically acceptable excipients for modifying or maintaining the pH, osmolarity, viscosity, sterility or stability of the pharmaceutical composition. The pharmaceutical compositions of the invention may contain still other pharmaceutically-acceptable formulation agents for modifying or maintaining the rate of release of a peptide of the invention. Such formulation agents include those substances known to the skilled Artisan in preparing sustained release formulations.

The pharmaceutical composition of this invention may be administered by any suitable means, such as parenterally (including intravenous, intramuscularly, subcutaneous, intra-arterial, intraperitoneal and intralesional); orally (including ingestion, buccal, or sublingual), by inhalation; intradermally; transdermally (topical); intracavity, intracranially, transmucosally or rectally.

A pharmaceutical composition may be stored in a sterile vial as a solution, suspension, gel, emulsion, solid, or dehydrated or lyophilized powder. Such compositions may be stored either in a ready to use form, a lyophilized form requiring reconstitution prior to use, a liquid form requiring dilution prior to use, or other acceptable form. In some embodiments, a pharmaceutical composition is provided in a single-use container (e.g., a single-use vial, ampoule, syringe, or autoinjector, whereas a multi-use container (e.g., a multi-use vial) is provided in other embodiments. Any drug delivery apparatus may be used to deliver the peptides of the invention, including implants (e.g., implantable pumps) and catheter systems, both of which are known to the skilled artisan. Depot injections, which are generally administered subcutaneously or intramuscularly, may also be utilized to release the peptides of the invention over a defined period of time. Depot injections are usually either solid- or oil-based and generally comprise at least one of the formulation components set forth herein.

Pharmaceutical compositions may be in the form of a sterile injectable aqueous or oleagenous suspension. This suspension may be formulated using suitable dispersing or wetting agents and suspending agents disclosed herein or known to the skilled Artisan. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose, any bland fixed oil may be employed including synthetic mono- or diglycerides. Moreover, fatty acids such as oleic acid find use in the preparation of injectable pharmaceutical compositions. Prolonged absorption of particular injectable formulations can be achieved by including an agent that delays absorption (e.g., aluminum monostearate or gelatin).

Although in general peptides are less suitable for oral administration due to susceptibility to digestion by gastric acids or intestinal enzymes, the compositions of the present invention may be administered orally due to the high activity observed for the stable retro-inverso peptides of the invention. In addition, novel methods are being used in order to design and provide metabolically stable and orally bioavailable peptidomimetic analogues.

Tablets, capsules and the like suitable for oral administration may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for preparation of such formulations are known to those skilled in the art and are commercially available.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by addition of water provide the active ingredient in admixture with a dispersing or wetting agent, suspending agent and one or more preservatives. Suitable dispersing or wetting agents and suspending agents are exemplified herein.

Pharmaceutical compositions of the invention may also be in the form of oil-in-water emulsions. The oily phase may be a vegetable oil, for example olive oil or arachis oil, or a mineral oil, for example, liquid paraffin, or mixtures of these. Suitable emulsifying agents may be naturally-occurring gums, for example, gum acacia or gum tragacanth; naturally-occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Pharmaceutical compositions can also include carriers to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, may be employed. Prolonged absorption of injectable pharmaceutical compositions can be achieved by including an agent that delays absorption, for example, aluminum monostearate or gelatin. Prevention of the action of microorganisms can be achieved by various antibacterial and antifungal agents, for example, parabens, chlorobutanol, phenol, ascorbic acid, thimerosal, and the like.

The invention also includes pharmaceutical compositions comprising the peptides of the invention peptides in the form of suppositories for rectal administration. The suppositories can be prepared by mixing a peptide of the invention with a suitable non-irritating excipient which is solid at ordinary temperatures but liquid at the rectal temperature and will therefore melt in the rectum to release the drug. Such materials include, but are not limited to, cocoa butter and polyethylene glycols.

The amount of a compound of the invention that will be effective in the treatment of a particular condition of diabetes will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the diabetic condition, and should be decided according to the judgment of the practitioner and each patient's circumstances. Effective doses may be extrapolated from dose-response curves derived from in vitro or animal model test bioassays or systems. According to certain embodiments, the doses can be extrapolated from the dose effective in treating mice. According to certain embodiments the amount to be intravenously administered to a human subject is the range of from about 1-100, 1-75, 1-50, 1-25, 1-15, 1-10, 1-5 mg/Kg human body weight. Each possibility represents a separate embodiment of the present invention. According to certain currently exemplary embodiments, the peptide is administered at 1-5 mg/Kg human body weight.

According to certain embodiments, the pharmaceutical composition comprising the peptides of the invention is administered at least once a day. According to some embodiments, the pharmaceutical composition is administered once a day. According to other embodiments, the pharmaceutical composition is administered once a week.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Material and Methods

### Materials

4′,6-diamidino-2-phenylindole (DAPI), Tris, trisodium, streptozotocin, Triton X-100, and Tween-20 were purchased from Sigma (St. Louis, Mo.). Eosin, hematoxylin, and oil red O were purchased from Fisher Scientific (Geel, Belgium). Dimethyl sulfoxide (DMSO) was purchased from MP Biomedicals (Solon, Ohio). Formaldehyde was purchased from Emsdiasum (Hatfield, Pa.). 3,3-diaminobenzidine (DAB) was obtained from ImmPact-DAB (Burlingame, Calif.), and Performa Accu-Chek glucometer and test strips (Roche, Indianapolis, Ind.). Dulbecco's modified Eagle's medium (DMEM), was purchased from Gibco (Grand Island, N.Y.). The sources and dilutions of primary and secondary antibodies are detailed in Table 1. R-Tf-D-LP4 peptide (KWTWK NSNGATWALNVATELKK-199-EWTWSHRPYIAH, SEQ ID NO:14), comprising 34 residues in D configuration (except the underlined Tf sequence) was synthesized by GL Biochem (Shanghai, China) to >95% purity. The R-Tf-D-LP4 peptide was first dissolved in DMSO as a 40 mM solution and then diluted 20-fold in the appropriate buffer. R-Tf-D-LP4 peptide concentration was determined using absorbance at 280 nm and the specific molar excitation coefficient.

TABLE 1
Antibodies used in the experiments of the present invention

| | | Dilution |
|---|---|---|
| Antibody | Source and Catalog No. | IHC, IF |
| Mouse anti-Glucagon | Abcam, Cambridge, UK, ab10988 | 1:500 |
| Mouse anti-insulin | Abcam, Cambridge, UK, ab6995 | 1:300 |
| Rabbit anti-Ki-67 | Abcam, Cambridge, UK, ab15580 | 1:250 |
| Rabbit anti-PDX1 | Abcam, Cambridge, UK, ab47267 | 1:300 |
| Rabbit anti-VDAC1 | Abcam, Cambridge, UK, ab15895 | 1:500 |
| Donkey anti-Mouse Alexa | Abcam, Cambridge, UK, ab150109 | 1:500 |
| Fluor 488 | | |
| Goat anti-Rabbit Alexa | Abcam, Cambridge, UK, ab150086 | 1:500 |
| Fluor 555 | | |
| Donkey anti-Mouse HRP | Abcam, Cambridge, UK, ab98799 | 1:500 |
| conjugated | | |
| Goat anti-Rabbit HRP | Promega, Madison, WI, W401B | 1:500 |
| conjugated | | |
| Mouse anti transporter | Abcam, Cambridge, UK, ab85715 | 1:500 |
| GLUT2 | | |

### Dietary Intervention

The HFD-32 diet was as described previously (Fujii, M., et al. Med Mol Morphol, 2013. 46(3): p. 141-52; 507.6 kcal/100 g, 56.7% kcal from fat) comprising 5% egg white powder (MM Ingredients, Wimborne, UK), 6.928% lactose (Pharma Grade, Nelson, UK), 15.88% beef fat (contains 80% fat), 5% AIN93G-mineral mixture, 0.002% tertiary butyl hydroquinone and 1.4% AIN93VX-vitamin mix (MP Biomedical, Illkirch, France), 24.5% milk casein (Shaanxi Fuheng Biotechnology, Xi'an, China), 20% safflower oil (high oleic acid content) (Bustan Briut, Galil, Israel), 6.45% sucrose, 0.43% L-cysteine, 5.5% crystalline cellulose (Sigma), 0.36% choline bitartrate and 8.25% maltodextrin (Bulk Powders, Colchester, UK). Control C57BL/6 mice were fed with a standard chow diet (408.4 kcal/100 g, 57% kcal from carbohydrates, 27% kcal from proteins, 16% from fat (V1154-703, S sniff Spezialdaten, Sosset, Germany).

### Ob/Ob Diabetes Mouse Model

Six week old male (C57BL/6) Ob/Ob (Jackson Laboratory, Bar Harbor, Me.) mice were treated with R-Tf-D-LP4 (14 mg/Kg) from week 6 to week 13 by intravenous administration of the R-Tf-D-LP4 peptide in 100 μl of Hank's balanced salts solution (HBSS) without calcium three times a week. A control group received 100 μl of 0.8% DMSO in HBSS buffer injected intravenously. The final blood DMSO concentration was 0.07% in both control and peptide-treated mice. Blood glucose levels were measured once a week. At the end of the experiment, the mice were sacrificed, and the pancreas was removed and fixed with formaldehyde, embedded in paraffin, sectioned, and subjected to immunefluorescent (IF). The experimental protocol used in the mouse model was approved by the Institutional Animal Care and Use Committee.

### NAFLD/Diabetes Mice Model

Male C57BL/6 mice were purchased from Envigo (Jerusalem, Israel). In order to generate the diabetes-based steatosis-NASH mouse model, 2-day-old mice were subjected to a single, low dose subcutaneous injection of streptozotocin (STZ) (200 μg/mouse) and then were fed with the HFD-32 high fat diet starting at the end of week 4. The mice manifested steatosis at 6 weeks and NASH at 8 weeks. These mice are designated herein interchangeably as “NAFLD/diabetes mice model” and “STZ/HFD-32-fed mice”. Mice affected with steatosis were treated with R-Tf-D-LP4 (14 mg/kg) from week 6 to week 8, and mice affected with NASH from week 8 to week 10. The R-Tf-D-LP4 peptide at 14 mg/kg (in 100 μl of Hank's balanced salts solution (HBSS) without calcium) was administered intravenously three times a week. Control groups were intravenously injected with 100 μl of 0.8% DMSO in HBSS buffer from week 6 to week 8 for steatosis affected mice and from week 8 to week 12 for NASH affected mice. The final blood DMSO concentration was 0.07% in control and peptide-treated mice.

At the end of the experiment, the mice were sacrificed by CO₂ inhalation and the livers and pancreases were removed. Livers were photographed and the lipid content was assessed. For this purpose, part of the liver was frozen in OCT (optimal cutting temperature) compound, embedded, sectioned, and stained with oil red O. The pancreases were fixed with formaldehyde, embedded in paraffin, sectioned, and subjected to immunohistochemical (IHC) or immunefluorescent (IF) staining, as described below.

The experimental protocol used in the mouse model was approved by the Institutional Animal Care and Use Committee.

Cell Culture and Treatment with R-Tf-D-LP4 Peptide

3T3-L1 mouse embryo fibroblasts were purchased from the American Type Culture Collection (ATCC Manassas, Va.) and maintained in a humidified atmosphere of 5% CO₂, 95% air at 37° C. in Dulbecco's modified Eagle's growth medium (DMEM) with high glucose (4.5 mM), supplemented with 10% fetal calf serum, and 1% penicillin-streptomycin. For R-Tf-D-LP4 peptide treatment, 3T3-F442A cells (6×10⁵/ml at 70-80% confluence) were incubated with the R-Tf-D-LP4 peptide in 500 μl serum-free medium for 24 h at 37° C. and 5% CO₂. The cells were then stained with Oil red O as described below.

### Oil Red O Staining

Following sacrifice, the mice livers were removed, and isolated livers were immediately embedded in OCT medium and kept at −80° C. until sectioning. Sections (10 μm thick) were washed with PBS, fixed with 4% formaldehyde for 10 min, gently washed with 60% isopropanol, and then stained with a solution of 0.5 g oil red O in 60% isopropanol for 15 min. The stained sections were washed several times with distilled water to remove unbound dye. The samples were then counter-stained with hematoxylin for 5 min and images were collected using a light microscope (Leica DM2500) with the same light intensity and exposure time.

3T3-L1 cells were washed with PBS, fixed with formaldehyde for 15 min, and then subjected to Oil Red O staining for 30 min at room temperature followed by staining with Hematoxylin. Microscope images were collected to visualize the red oil droplets in the cells. Finally, the cells were washed with H₂O followed by three washes with 60% isopropanol with gentle rocking. The Oil Red O stain was extracted with 100% isopropanol for 5 min and the absorbance at 492 nm was measured and normalized to the same number of cells counted before stain extraction.

### Immunohistochemistry and Immunofluorescence Analysis of Pancreas Tissue

Immunohistochemistry (IHC) and immunofluorescence (IF) staining was performed on 5 μm-thick formalin-fixed and paraffin-embedded pancreatic tissue sections. Sections were deparaffinized (5 minutes in xylene, 3 times), followed by rehydration with a graded ethanol series (50%, 70%, 95%, 100%). Antigen retrieval was performed by 20 min incubation in preheated 0.01 M citrate buffer, pH 6.0 at 95-98° C. Sections were washed with PBS pH 7.4 containing 0.1% Triton-X100 (PBST), incubated in 10% NGS and 1% BSA for 2 h, followed by overnight incubation at 4° C. with primary antibodies (see Table 1). Sections were then washed with PBST. For IHC staining, endogenous peroxidase activity was blocked by incubating the sections in 3% H₂O₂ for 15 min. Following washing with PBST, sections were incubated for 2 h with the appropriate secondary HRP-conjugated antibodies. Sections were washed with PBST and peroxidase activity was visualized by incubating with DAB. After rinsing in water, the sections were counter-stained with hematoxylin, dehydrated with a graded ethanol series (50-100%), incubated in xylene, and mounted with mounting medium. Finally, images from the sections were collected using an automatic digital slide scanner (Panoramic

MIDI II, 3DHISTECH) with the same light intensity and exposure time. Non-specific control experiments were carried out using the same protocols but omitting incubation with primary antibodies. For IF staining, sections were incubated for 2 h with the appropriate secondary Alexa Fluor conjugated antibodies. Sections were washed with PBST, counter—stained with DAPI (0.07 μg/ml), washed with PBST, and mounted with mounting media. The slides were then viewed with an Olympus IX81 confocal microscope or scanned using an automatic digital slide scanner.

### Blood Glucose Measurement

Blood was collected in a capillary tube by retro orbital bleeding as described previously (Amrani, A., et al., Endocrinology, 1998. 139(3): p. 1115-24) from the

STZ/HFD-32-fed mice at the end of study before sacrifice. In the case of the ob/ob mice, weekly blood samples were collected from the tail, and blood glucose levels were measured immediately using an Accu-Check Performa blood glucose meter.

### Statistics and Data Analysis

The mean±SEM of results obtained from at least two independent experiments with the data derived from several mice in each experiment (n=5-8 for each group) are presented. One-way analysis of variance (ANOVA) followed by Mann-Whitney post hoc test using Graphpad Prism 6 was employed to evaluate significant differences between the experimental groups. P values were: *p<0.05, **p<0.01, ***p<0.001, ****p<

### Example 1: Effects of R-Tf-D-LP4 Peptide on Blood Glucose Level and on Langerhans Islets in ob/ob Mice

The ob/ob, leptin-deficient mouse, is a commonly used murine model for diabetes and obesity. ob/ob mice are hyperglycemic, hyperinsulinemic, hyperlipidemic, and insulin resistant (King, A. J., Br J Pharmacol, 2012. 166(3): p. 877-94; Lindstrom, P., Scientific World Journal, 2007. 7: p. 666-85), which makes them suitable for evaluating the effects of various factors on obesity and hyperglycemia.

In STZ/HFD-32-fed mice, the R-Tf-D-LP4 peptide accelerated β-oxidation in the liver (Pittala et al., 2019 ibid). Thus, the R-Tf-D-LP4 peptide is expected to affect the weight of the ob/ob mice. However, no such effect was observed, and the weekly average weight was found to be similar whether or not the ob/ob mice were treated with R-Tf-D-LP4 peptide (FIG. 1B). Since it was possible that the R-Tf-D-LP4 peptide could not reach the fat tissue, 3T3-F442A adipocyte cell-line were used to test the effect of the R-Tf-D-LP4 peptide on the storage of intracellular lipids as visualized by Oil Red O staining (FIG. 1C). The results showed that treatment with 10 μM R-Tf-D-LP4 peptide decreased the amount of lipid in 3T3-L1 adipocytes by 90% as quantified by extraction of Oil red O stain (FIG. 1D). It is therefore likely that the R-Tf-D-LP4 peptide either accelerates fatty acid oxidation or inhibits fatty acid synthesis or both as found for the STZ/HFD-32 fed mice (Pittala et al., 2019 ibid).

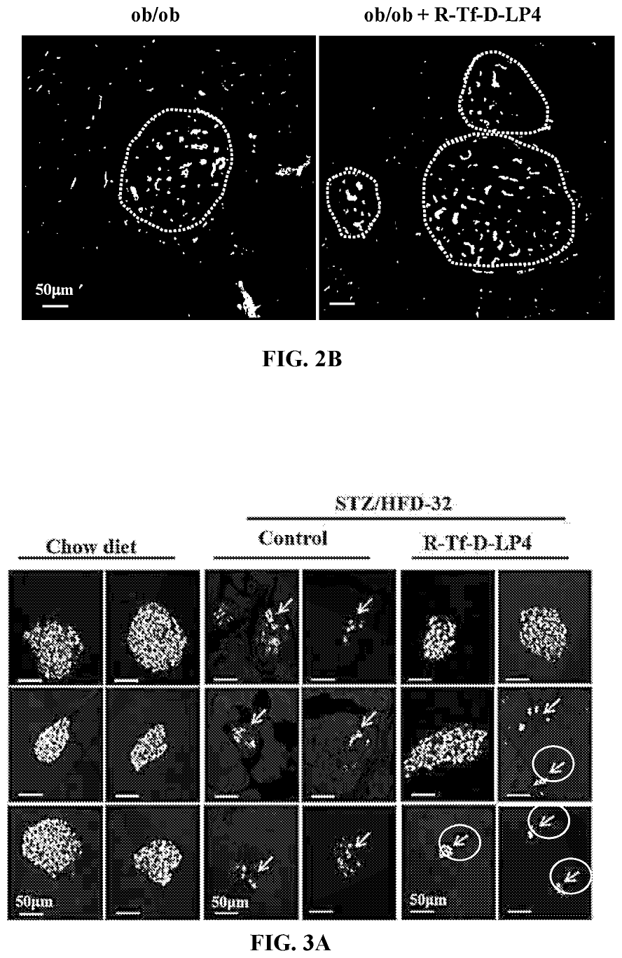

The effects of the R-Tf-D-LP4 peptide on Langerhans islets of the ob/ob mice were examined by hematoxylin and Eosin (FIG. 2A) and anti-insulin antibodies (FIG. 2B) staining of paraffin sections of the pancreas. As described previously (Lindstrom, 2007, ibid; Bock, T., B. Pakkenberg, and K. Buschard, Diabetes, 2003. 52(7): p. 1716-22), the size of the islets was significantly increased in the pancreas of ob/ob mice (FIG. 2) compared to islets of wild type (WT) mice (FIG. 2). Treatment of ob/ob mice with the R-Tf-D-LP4 peptide increased the number of islets, as was also found for STZ/HFD-32 mice treated with R-Tf-D-LP4 peptide (see Example 2 and FIG. 3, Table 2), and thereby also improved insulin production. This may explain the reduction in blood glucose level induced by the peptide.

### Example 2: Effect of Treatment with R-Tf-D-LP4 Peptide on Pancreatic Endocrine Activity in STZ/HFD-32-Fed Mice

In order to evaluate the endocrine activity of Langerhans islets in regulating blood glucose in NAFLD/diabetes mice model, the size and numbers of islets, as visualized by IF staining for insulin in the steatosis state (FIG. 3A), and by H & E staining in the NASH stage (FIG. 3B), compared to chow-diet-fed mice was analyzed. As expected, there were large clusters of islets stained with anti-insulin antibody in the pancreatic sections obtained from control animals fed with chow diet. (FIG. 3). However, no such clusters were observed in the pancreases from STZ/HFD-32-fed mice at either the steatosis (FIG. 3A) or NASH (FIG. 3B) stage, and instead the islets appeared disrupted (FIG. 3A, arrows). After treatment with R-Tf-D-LP4 peptide, both the size and the number of Langerhans islets were increased. The results from several experiments with and without R-Tf-D-LP4 peptide treatment are summarized in Table 2. Langerhans islets were counted in each pancreas and categorized into several groups according to their size. Overall, the number of Langerhans islets in pancreases of STZ/HFD-32 fed mice in the steatosis or NASH stage was decreased by 50% and 40%, respectively, relative to the values in the chow diet-fed mice. R-Tf-D-LP4 peptide treatment increased the number of the islets in mice with steatosis to the level observed in mice fed with chow diet, and further increased the number in mice affected with NASH by 30%. An increase in both large and small islets was observed in the sections from mice treated with R-Tf-D-LP4 peptide although most of the increase was in the small islets (FIG. 3A, circled arrows, and Table 2).

TABLE 2
Effect of the R-Tf-D-LP4 peptide on the number of Langerhans
islets in STZ/HFD-32 diet fed mice with steatosis or NASH

| | Steatosis | | | | |
|---|---|---|---|---|---|
| Parameter | Chow | HFD-32 | | HFD-32, Peptide | |
| Total No. of islets | 136 (n = 5) | 217 (n = 16) | | 451 (n = 17) | |
| Average No. of islets per mouse | 27 | 14 | (<0.001) | 27 | (ns) |
| Islet size, 1-50 nm | 11 | 4 | (<0.05) | 16 | (ns) |
| Islet size, 51-100 nm | 8 | 6 | (<0.05) | 8 | (ns) |
| Islet size, 101-150 nm | 5 (ns) | 3 | (ns) | 2 | (ns) |
| Islet size, 151-300 nm | 3 | 1 | (<0.05) | 1 | (ns) |

| | NASH | | | | |
|---|---|---|---|---|---|
| Parameter | Chow | HFD-32 | | HFD-32, Peptide | |
| Total No. of islets | 136 (n = 5) | 168 (n = 10) | | 354 (n = 10) | |
| Average No. of islets per mouse | 27 | 17 | (<0.001) | 35 | (<0.05) |
| Islet size, 1-50 nm | 11 | 8 | (ns) | 16 | (ns) |
| Islet size, 51-100 nm | 8 | 6 | (ns) | 12 | (ns) |
| Islet size, 101-150 nm | 5 | 2 | (ns) | 4 | (ns) |
| Islet size, 151-300 nm | 3 | 1 | (ns) | 3 | (ns) |

### Example 3: Effect of R-Tf-D-LP4 Peptide on the Morphology of Langerhans Islets

Morphology of normal islet of Langerhans with the glucagon producing α-cells present around the edge could be seen in glucagon IHC staining of the mice fed with chow diet (FIG. 4A). However, in the STZ/HFD-32-fed mice with steatosis or NASH, the islets demonstrated an impaired morphology in which the glucagon-producing cells infiltrated into the islets (FIG. 4A, 4B). This was improved to some extent in islets from the STZ/HFD-32-fed mice treated with R-Tf-D-LP4 at the steatosis stage (FIG. 4A) but was completely restored in animals treated with R-Tf-D-LP4 peptide at the NASH stage, where, the glucagon stained a—cells were localized as expected around the edge of the islet (FIG. 4B).

Glut-2 encoded by SLC2A2, is predominantly expressed in hepatocytes, but also in kidney proximal convoluted tubule cells, intestinal absorptive cells, and pancreatic β-cells (Kellett, G. L., et al., Annu Rev Nutr, 2008. 28: p. 35-54; Cramer, S.C., et al., Diabetes, 1992. 41(6): p. 766-70). Glut-2 is involved in glucose-sensing in pancreatic β-cells, liver, and the hypothalamus, as well as in triggering the glucose-mediated insulin secretion cascade (Dupuis, J., et al., Nat Genet, 2010. 42(2): p. 105-16). Glut-2 is thought to be involved in the pathogenesis of diabetes mellitus. Studies have reported that in diabetic animal models, Glut-2 expression is down-regulated in pancreatic β-cells (Bonny, C., et al., Mol Cell Endocrinol, 1997. 135(1): p. 59-65), while hepatic expression of this glucose transporter is enhanced (Okamoto, Y., S. Tanaka, and Y. Haga, Hepatol Res, 2002. 23(2): p. 138-144). In addition, mice lacking Glut-2 developed early diabetes and abnormal postnatal pancreatic islets (Guillam, M. T., et al., Nat Genet, 1997. 17(3): p. 327-30), and loss of sugar detection by Glut-2 affects glucose homeostasis (Stolarczyk, E., et al., PLoS One, 2007. 2(12): p. e1288).

Here, the expression of Glut-2 was analyzed in the pancreas of STZ/HFD-32-fed mice which received/did not receive the R-Tf-D-LP4 peptide treatment (FIG. 4C). Glut-2 was present in the islets of the chow diet-fed mice, was not detected in the STZ/HFD-32 fed mice but was re-expressed when the mice were treated with R-Tf-D-LP4 peptide (FIG.

4C).

### Example 4: VDAC1 Expression Levels in Langerhans Islets of STZ/HFD-32-Fed Mice and the Effect of R-Tf-D-LP4 on the Expression

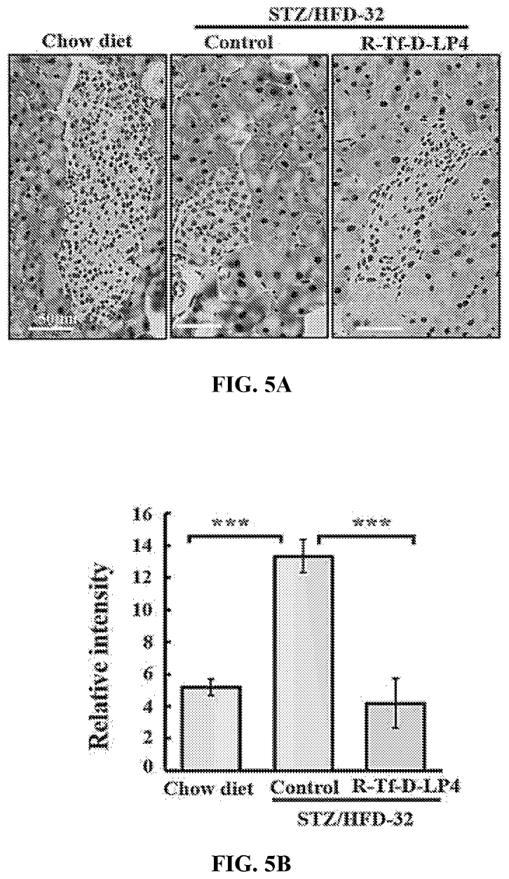

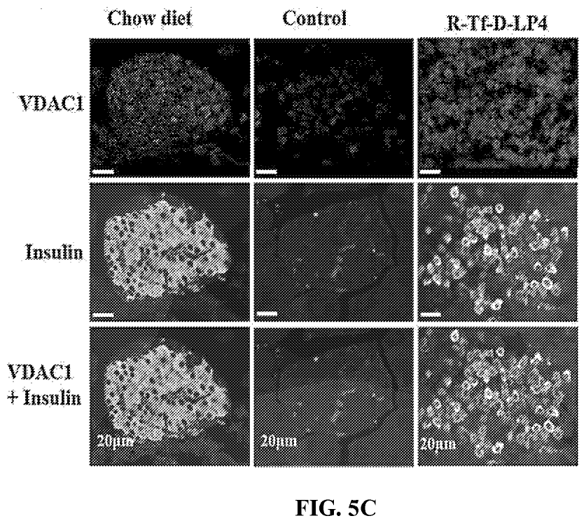

It has been previously demonstrated that the level of VDAC1 is upregulated in islets of donors diagnosed with T2D and in control islets under conditions of glucotoxicity, as well as in the T2D mouse model (Zhang et al., 2019, ibid), and in livers of the STZ/HFD-32-fed mouse model (Pitalla et al., 2019, ibid). The expression of VDAC1 was examined in the pancreas of STZ/HFD-32 diet-fed mice, representing NAFLD affected mice on a diabetic background with and without R-Tf-D-LP4 peptide treatment as compared to the levels in islets from mice fed with chow-diet (FIG. 5). Formaldehyde-fixed, paraffin-embedded pancreas sections were analyzed by IHC staining for VDAC1 using anti-VDAC1 specific antibodies. The results demonstrated increased staining in the islets of NAFLD mice relative to chow-diet fed mice (FIG. 5A). However, this level decreased (FIG. 4A) in mice treated with R-Tf-D-LP4 peptide. In addition, the size of the islets was smaller in STZ/HFD-32-fed mice than in the chow-diet fed mice or in STZ/HFD-32-fed mice treated with R-Tf-D-LP4 (FIG. 5A). A quantitative analysis of the staining intensity in the islets revealed a significant increase in VDAC1 staining in the STZ/HFD-32-fed mice as compared to both chow-diet fed mice and STZ/HFD-32-fed mice treated with R-Tf-D-LP4 peptide (FIG. 5B).

Similar results were obtained using IF and co-staining for VDAC1 and insulin expression, to better identify the insulin producing islets. In this case, the results showed that the islets were disrupted and smaller in the STZ/HFD-32-fed mice than in the controls, but that mice treated with the R-Tf-D-LP4 peptide displayed a normal morphology and large islets (FIG. 5C).

### Example 5: Effect of R-Tf-D-LP4 Treatment on Proliferation of Islet Cells

A possible explanation for the increase in islet size and number is the proliferation/regeneration of β-cells or other cell types comprised in Langerhans islets such as α-cells. A known marker for cell proliferation is the Ki-67 protein. Accordingly, to evaluate proliferation, pancreatic sections were IF stained using specific anti-Ki-67 antibodies. Counting the Ki-67 positive cells in the pancreatic sections from mice fed with chow diet and from STZ/HFD-32-fed mice, revealed similarly low numbers and distribution of the Ki-67 positive cells inside and in the periphery of the islets (FIG. 6A, white arrows). However, R-Tf-D-LP4-treated mice showed that the number of Ki-67 positive cells increased both in the islet interior and in cells outside of the islets (FIG. 6A, arrows) by about 8-fold and 3-fold respectively, compared to both chow diet-fed mice and STZ/HFD-32-fed mice (FIG. 6B).

### Example 6: Effect of Treatment with R-Tf-D-LP4 Peptide on β- and α-Cell Development

Another possible explanation for the increase in the number and size of insulin producing cells in the Langerhans islets following R-Tf-D-LP4 peptide treatment is the differentiation of precursor cells or the trans-differentiation of differentiated cells such as α-cells into insulin producing β-cells. One of the markers of β-cell maturation and a necessary factor for pancreatic development is PDX1 (pancreatic and duodenal homeobox 1) also known as insulin promoter factor 1. This is a transcription factor that, among other functions, enhances the expression of the insulin encoding gene (INS) (Ohlsson, H., K. Karlsson, and T. Edlund, EMBO J, 1993. 12(11): p. 4251-9). In order to assess the impact of treatment with R-Tf-D-LP4 peptide on PDX1 expression in β-cells and α-cells, pancreatic sections were co-stained for PDX1 and insulin (FIG. 7A) or glucagon (FIG. 7B), using specific antibodies. The high level of PDX1 staining in the pancreases from chow diet-fed mice was strongly decreased in STZ/HFD-32-fed mice (FIG. 7A). In contrast, pancreases obtained from STZ/HFD-32-fed mice treated with R-Tf-D-LP4 peptide showed a higher level of staining for PDX1, with some, but not all, of the cells co-stained for insulin (FIG. 7A).

Glucagon staining in chow diet-fed mice showed the expected staining in the islet periphery, but the staining tended to be in the islet interior in STZ/HFD-32-fed mice, with and without R-Tf-D-LP4 peptide treatment (FIG. 7B) as shown above for glucagon staining (FIG. 6A). As already discussed, PDX1 staining was high in chow diet-fed mice, strongly decreased in STZ/HFD-32-fed mice, and was highly increased in mice treated with R-Tf-D-LP4 peptide over the level in the control chow-fed mice (FIG. 7B). These results suggest that R-Tf-D-LP4 peptide treatment increases the expression of the transcription factor PDX1 thereby enhancing the expression of the insulin encoding gene.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-42. (canceled)

43. A method for treating diabetes and/or preventing the progress of diabetes, comprising administering to a subject affected with prediabetes or with diabetes a therapeutically effective amount of a pharmaceutical composition comprising at least one synthetic peptide comprising a retro modified and partially or completely inverso modified analogue of a VDAC1-derived peptide, wherein the VDAC1-derived peptide comprises an amino acid sequence selected from the group consisting of SEQ ID NO:1 and SEQ ID NO:2.

44. The method of claim 43, wherein the subject having diabetes is newly diagnosed early after onset of the disease.

45. The method of claim 43, wherein the diabetes is selected from the group consisting of Type 1 diabetes, Type 2 diabetes and gestational diabetes.

46. The method of claim 43, wherein at least one exists:
a. the subject has a fasting plasma glucose (FPG) level selected from the group consisting of greater than about 100 mg/dl; between about 100 and 130 mg/dl; and greater than about 130 mg/dl;
b. the subject has FPG level of greater than about 200 mg/dl; or
c. the subject has a hemoglobin A1c (HbA1c) level above about 6%.

47. The method of claim 46, wherein treating and/or preventing the progress of diabetes comprises reducing the FPG level and/or the HbA1c level of the subject by at least 5% compared to the FPG and/or HbA1c level measured before administration of the pharmaceutical composition.

48. The method of claim 43, wherein treating and/or preventing the progress of diabetes comprises at least one of preserving pancreatic islets number and/or size and/or function; preventing pancreatic islet degeneration and/or dysfunction; restoring insulin secretion from pancreatic islet β-cells; inducing glucose-stimulated insulin secretion; restoring the number of functional pancreatic islets to a normal level; and any combination thereof.

49. The method of claim 43, wherein the subject is a human subject selected from the group consisting of pre-pubertal child, post-pubertal child, adolescent and adult.

50. The method of claim 43, wherein the pharmaceutical composition is formulated for administration by a route selected from the group consisting of parenteral, oral, transdermal, topical, intranasal, or via a suppository administration.

51. The method of claim 50, wherein the pharmaceutical composition is formulated for intravenous administration, and wherein said pharmaceutical composition is administered to a human subject at an amount of the synthetic peptide of from about 1 to about 100 mg/Kg human body weight.

52. The method of claims 43, wherein the analogue of the VDAC1-derived peptide comprises the amino acid sequence selected from the group consisting of SEQ ID NO:3 and SEQ ID NO:5.

53. The method of claim 43, wherein the analogue of VDAC1-derived peptide is completely inverso modified.

54. The method of claim 43, wherein the synthetic peptide further comprises a cell recognition and/or localization moiety.

55. The method of claim 54, wherein the cell recognition and/or localization moiety is a peptide selected from an all L-stereoisomeric peptide and an all D-stereoisomeric peptide.

56. The method of claim 55, wherein the recognition and/or localization peptide comprises a transferrin-receptor binding domain (Tf), a fragment thereof or a retro-analog of same.

57. The method of claim 56, wherein the transferrin-receptor binding domain comprises the amino acid sequence set forth in SEQ ID NO:7.

58. The method of claim 55, wherein the retro-analogue of the transferrin-receptor binding domain (Tf) has the amino acid sequence set forth in SEQ ID NO:8, and wherein all said amino acids are in L configuration.

59. The method of claim 54, wherein the recognition and/or localization moiety is connected to the N- or the C-terminus of the analogue of VDAC1-derived peptide directly or via a linker sequence.

60. The method of claim 54, wherein the synthetic peptide further comprises the amino acid sequences set forth in SEQ ID NO:11 and the amino acid sequence set forth in SEQ ID NO:12, each independently located at the C- or N-terminus of the analogue of VDAC1-derived peptide.

61. The method of claim 54, wherein the synthetic peptide further comprises the amino acid sequences set forth in SEQ ID NO:12 and the amino acid sequence set forth in SEQ ID NO:13, each independently located at the C- or N-terminus of the analogue of VDAC1-derived peptide.

62. The method of claim 61, wherein the synthetic peptide comprises the amino acid sequence set forth in SEQ ID NO:14.


SEQUENCE LISTING (16 sequences)

SEQ ID NO: 1 - protein, 17 aa, Artificial Sequence
  Peptide
  misc_feature: Synthetic
KKLETAVNLAWTAGNSN

SEQ ID NO: 2 - protein, 26 aa, Artificial Sequence
  Peptide
  misc_feature: Synthetic
MAVPPTYADLGKSARDVFTKGYGFGL

SEQ ID NO: 3 - protein, 17 aa, Artificial Sequence
  Peptide
  misc_feature: Synthetic
NSNGATWALNVATELKK

SEQ ID NO: 4 - protein, 17 aa, Artificial Sequence
  Peptide. All amino acids are in the D-configuration.
  misc_feature: Synthetic
NSNGATWALNVATELKK

SEQ ID NO: 5 - protein, 26 aa, Artificial Sequence
  Peptide
  misc_feature: Synthetic
LGFGYGKTFVDRASKGLDAYTPPVAM

SEQ ID NO: 6 - protein, 26 aa, Artificial Sequence
  Peptide. All amino acids are in the D-configuration form.
  misc_feature: Synthetic
LGFGYGKTFVDRASKGLDAYTPPVAM

SEQ ID NO: 7 - protein, 7 aa, Artificial Sequence
  Peptide
  misc_feature: Synthetic
HAIYPRH

SEQ ID NO: 8 - protein, 7 aa, Artificial Sequence
  Peptide
  misc_feature: Synthetic
HRPYIAH

SEQ ID NO: 9 - protein, 16 aa, Artificial Sequence
  Peptide
  misc_feature: Synthetic
RQIKIWFQNRRMKWKK

SEQ ID NO: 10 - protein, 16 aa, Artificial Sequence
  Peptide
  misc_feature: Synthetic
KKWKMRRNQFWIKIQR

SEQ ID NO: 11 - protein, 5 aa, Artificial Sequence
  Peptide
  misc_feature: Synthetic
SWTWE

SEQ ID NO: 12 - protein, 5 aa, Artificial Sequence
  peptide
  misc_feature: Synthetic
KWTWK

SEQ ID NO: 13 - protein, 5 aa, Artificial Sequence
  Peptide
  misc_feature: Synthetic
EWTWS

SEQ ID NO: 14 - protein, 34 aa, Artificial Sequence
  Peptide
  misc_feature: Synthetic
  MISC_FEATURE (1)..(27): Amino acids in the D configuration form
KWTWKNSNGATWALNVATELKKEWTWSHRPYIAH

SEQ ID NO: 15 - protein, 42 aa, Artificial Sequence
  Peptide
  misc_feature: Synthetic
  MISC_FEATURE (17)..(42): Amino acids in the D configuration form
KKWKMRRNQFWIKIQRLGFGYGKTFVDRASKGLDAYTPPVAM

SEQ ID NO: 16 - protein, 34 aa, Artificial Sequence; identical to SEQ ID NO: 14
  Peptide
  misc_feature: Synthetic
  MISC_FEATURE (6)..(22): Amino acids in the D configuration form